(19) United States
(12) Patent Application Publication
(10) Pub. No.: US 2016/0183570 A1
(43) Pub. Date: Jun. 30, 2016

(54) PROCESSING TOMATO AND METHODS OF PRODUCING AND USING SAME

(71) Applicant: The St. of Israel, Ministry of Agriculture & Rural Dev., Agricultural Res. Org. (ARO)(Volcani Center, Beit-Dagan (IL)

(72) Inventor: Arthur A. Schaffer, Hashmonaim (IL)

(21) Appl. No.: 14/907,316

(22) PCT Filed: Jul. 24, 2014
(86) PCT No.: PCT/IL2014/050677

§ 371 Date: Jan. 25, 2016

Related U.S. Application Data
(60) Provisional application No. 61/858,159, filed on Jul. 25, 2013.

(51) Int. Cl. A23L 1/212; C12N 15/82; A01H 5/08; A01H 1/02

(52) U.S. Cl. CPC A23L 1/2128; A01H 5/08; A01H 1/02; C12N 15/8249; C12N 15/8246; A23V 2002/00

(57) ABSTRACT

A processing tomato fruit is provided. The processing tomato is characterized by having an intact skin and a Brix value higher than 10 under conditions for natural dehydration, the natural dehydration being generally unaccompanied by microbial spoilage.

### FIELD AND BACKGROUND OF THE INVENTION

The present invention, in some embodiments thereof, relates to a processing tomato and methods of producing and using same.

Tomatoes are used in the form of various processed products, including concentrated tomato pastes. The production of tomato pastes is dependent on the energy-requiring steps of concentration which is carried under by dehydration of the tomato pulp. Generally, tomato fruit and the pulp produced from them, which are used for the production of paste comprises between 90-95% water. The remainder of the pulp, without the portions that are excluded from paste, such as skin and seeds, which makes up generally between 4 and 6% of the pulp is referred to as the tomato solids.

Fresh tomatoes are generally harvested mechanically in a single harvest, which has been made possible by the breeding of determinate growth habit varieties with a concentrated harvest period. The window of harvest time is framed and limited by the need to reach a maximum of harvestable ripe fruit yield on the one hand but the fruit may not remain too long on the vine since standard tomato fruit will be susceptible to numerous problems including fruit rot.

In light of the costs and difficulties involved in transporting such a large bulk of harvested tomatoes, which is primarily water, from the field to the factory, together with the energy-requiring steps of dehydration and concentration upon arrival at the processing plant, there is a need for the development of naturally dehydrating tomatoes that may be used for the production of tomato paste. These tomatoes would naturally dehydrate while still attached to the vine in the field, or would dehydrate off the vine, thereby reducing volume for transportation and also reducing the energy-requiring dehydration in the processing plant.

Natural dehydrating tomatoes have been previously described (U.S. Pat. No. 7,119,261). These were developed by introgressing an allele for the cwp gene, derived from green-fruited wild species of tomato. The wild species allele is expressed in the developing fruit, causing the development of microfissures in the skin which allows for dehydration to naturally occur upon ripening. PCT Publication WO2006/030445 teaches the isolation and characterization of the cwp gene that increases the cuticular water permeability (CWP) of the mature red tomato fruit and leads to the dehydration of the intact fruit.

Determinate tomato varieties harboring the wild species cwp allele have been grown in the open field as industry tomatoes (PCT Publication WO 2008/119618) in an attempt to use these fruit to produce tomato paste. In WO 2008/119618 the tomatoes with a wild species cwp allele were allowed to remain on the vine for an unknown period of time past the normal harvest stage, accompanied by a modest increase in Brix value to about 7, as compared to the Brix value of about 5 at the normal harvest stage. Paste was produced from pulp of these modestly dehydrated tomatoes using standard industry methods for producing a paste of 16 Brix. These steps comprised evaporating the pulp mixture under heat (70° C.) in a vacuum chamber (EP2131662B1, [0073]). In addition, PCT Publication WO 2008/119618 describes the production of paste from such tomatoes which were harvested at the normal harvest stage of Brix ˜5 and then dehydrated in an oven to reach Brix of 14. Pulp from these tomatoes, following oven dehydration, was also used to produce paste of 16 Brix using standard methods for producing tomato paste.

Accordingly, the prior art describes paste produced from naturally, non-oven, dehydrated tomatoes that were of Brix of ˜7, as well as paste produced from oven-dried tomatoes which reached Brix higher than 7. The results of a taste panel indicated that the dried tomatoes which reached a Brix above ˜10 produced inferior tasting paste, making these tomatoes unsuitable for the production of paste.

### SUMMARY OF THE INVENTION

According to an aspect of some embodiments of the present invention there is provided a processing tomato fruit having an intact skin and a Brix value higher than 10 under conditions for natural dehydration, the natural dehydration being generally unaccompanied by microbial spoilage.

According to an aspect of some embodiments of the present invention there is provided a processing tomato fruit having an intact skin and a Brix value higher than 7.1 under conditions for natural dehydration, the natural dehydration being generally unaccompanied by microbial spoilage, wherein the conditions for natural dehydration comprise allowing the processing tomato fruit to remain on the vine post ripening for a time sufficient to reach the Brix value.

According to some embodiments of the invention, the conditions for natural dehydration comprise allowing the processing tomato fruit to remain on the vine post ripening for a time sufficient to reach the Brix value.

According to some embodiments of the invention, the conditions for natural dehydration comprise sun-drying prior to or following harvest of the processing tomato fruit.

According to some embodiments of the invention, the processing tomato is dehydrated.

According to some embodiments of the invention, the processing tomato is naturally dehydrated.

According to some embodiments of the invention, the processing tomato is fully ripe and not subjected to dehydration.

According to some embodiments of the invention, the processing tomato is unripe.

According to some embodiments of the invention, the natural dehydration is defined as wrinkling of the intact skin of the processing tomato fruit post ripening following harvesting.

According to some embodiments of the invention, the processing tomato characterized by an intact skin which permits dehydration of the fruit so as to obtain wrinkling of the skin.

According to some embodiments of the invention, the genome of the processing tomato fruit comprises a nucleic acid sequence encoding the cwp polypeptide, wherein the cwp polypeptide causes increased water permeability.

According to some embodiments of the invention, the nucleic acid sequence is under a transcriptional control of a heterologous promoter.

According to some embodiments of the invention, the nucleic acid sequence forms a part of an introgression derived from a wild Lycopersicon spp. the introgression comprising a portion of chromosome 4 of the Lycopersicon spp.

According to some embodiments of the invention, the portion of chromosome 4 of the Lycopersicon spp. is smaller than a chromosomal fraction extending from telomeric marker TG464 to centromeric marker CT173.

According to some embodiments of the invention, the Lycopersicon spp. is Lycopersicon hirsutum.

According to some embodiments of the invention, the processing tomato weighs 40-80 gr when fully ripe and prior to the natural dehydration.

According to some embodiments of the invention, the processing tomato weighs 60-80 gr when fully ripe and prior to the natural dehydration.

According to some embodiments of the invention, the Brix value is 12-30.

According to some embodiments of the invention, the Brix value is 12-25.

According to some embodiments of the invention, the Brix value is 12-20.

According to some embodiments of the invention, the processing tomato has a Brix value lower than 5 when fully ripe and prior to the natural dehydration.

According to an aspect of some embodiments of the present invention there is provided a plant comprising the fruit.

According to an aspect of some embodiments of the present invention there is provided a seed of the tomato fruit.

According to an aspect of some embodiments of the present invention there is provided a sown field comprising the seeds.

According to an aspect of some embodiments of the present invention there is provided a seed bag comprising the seeds.

According to an aspect of some embodiments of the present invention there is provided a method of producing a tomato paste comprising:

subjecting the processing tomato to natural dehydration so as to obtain dehydrated processing tomatoes; and subsequently comminuting or macerating the dehydrated processing tomatoes, thereby producing a tomato paste.

According to an aspect of some embodiments of the present invention there is provided a tomato paste generated according to the method.

According to an aspect of some embodiments of the present invention there is provided an edible processed tomato product comprising the processing tomato or an edible portion thereof.

According to some embodiments of the invention, the edible processed tomato product is selected from the group consisting of a tomato paste, a ketchup, a tomato sauce a tomato soup, a tomato juice, a tomato powder, a tomato dice, a crushed tomato, a chopped tomato and a tomato concentrate.

According to an aspect of some embodiments of the present invention there is provided a method of producing the tomato, the method comprising:

crossing a processing tomato plant with a wild Lycopersicon spp. so as to obtain hybrid plants; and

selecting plants of the hybrid plants having fruits exhibiting a Brix value above 7.1 when subjected to natural dehydration, thereby producing the tomato.

According to some embodiments of the invention, the method further comprises self crossing the hybrid plants and back-crossing with the processing tomato plants.

According to an aspect of some embodiments of the present invention there is provided a processing tomato fruit having a Brix value higher than 7.1 or 10 when subjected to natural dehydration, wherein representative sample of seeds of the processing tomato are deposited under the Budapest treaty at the NCIMB Ltd. (Ferguson Building Craibstone Estate, Bucksburn. Aberdeen Scotland) XXX (line 1027) or XXX (line 1028).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F are images of line 4 generated according to the teachings of the present invention.

FIG. 1G is an image of 4 different dehydrating tomatoes following natural dehydration. All weights are given for fully ripe tomato prior to dehydration. Top right (triplicate) line 3 fresh weight approx 50 g. Top left (triplicate) line 4 fresh weight approx 40 g. Bottom left (triplicate) line 5 fresh weight approx 30 g. Bottom right (triplicate) cherry variety of raisin tomato fresh weight approx 15 g. Lines 3-5 designate a tomato of a determinate habit.

FIG. 2 is an image of electrophoresis in 2% agarose gel showing PCR products from Habrochaites (248 bps) and from Lycopersicum (213 bps) exhibiting a 35 bps deletion.

### DESCRIPTION OF SPECIFIC EMBODIMENTS OF THE INVENTION

The present invention, in some embodiments thereof, relates to a processing tomato and methods of producing and using same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

U.S. consumption of processed tomato products has been on the rise over the past two decades. For the processing industry, much of the increased consumption is attributable to the increased presence of tomato products used in popular food items and condiments such as pizzas, pastas, salsa and ketchup.

In light of the costs and difficulties involved in transporting the large bulk of harvested processing tomatoes, which is primarily water, from the field to the factory, together with the energy-requiring steps of dehydration and concentration upon arrival at the processing plant, there is a need for the development of naturally dehydrating processing tomatoes that may be used for the production of processing tomato products.

Whilst reducing the present invention to practice, the present inventors developed a processing tomato which is characterized by a Brix value higher than 10 when allowed to naturally dehydrate at ambient temperatures (sun drying) without the use of oven drying. These tomatoes have been developed by cross-hybridization of L. esculentum with L. hirsutum. These tomatoes are characterized by the introgression of a wild species allele of cwp, allowing natural dehydration. The tomato fruits thus obtained reach high levels of dehydration and Brix values of the macerated pulp of over 7 and as high as 30. These partially dehydrated tomatoes can be used for the direct production of a concentrated tomato pulp with excellent taste. Processed products such as pastes generated from processing tomato fruits of some embodiments of the invention are characterized by levels of GABA, glutamine, proline, cysteine, methionine and total free amino acids which are higher than those commercially available, rendering them especially favorable in terms of nutritional value and taste.

Thus, according to an aspect of the invention there is provided a processing tomato fruit having an intact skin and a Brix value higher than 10 under conditions for natural dehydration, said natural dehydration being generally unaccompanied by microbial spoilage.

According to another aspect of the invention there is provided a processing tomato fruit having an intact skin and a Brix value higher than 7.1 under conditions o natural dehydration, said natural dehydration being generally unaccompanied by microbial spoilage, wherein said conditions for natural dehydration comprise allowing the processing tomato fruit to remain on the vine post ripening for a time sufficient to reach the Brix value.

As used herein the phrase “processing tomato” refers to a determinate tomato plant. According to some embodiments of the invention the plant has a fruit weight of about 30-80 gr/unit when fully ripe and prior to the natural dehydration. Other exemplary fruit weight ranges include, but are not limited to about, 30-40 gr, 35-80 gr/unit, 35-70 gr/unit, 40-70 gr/unit, 40-80 gr/unit, 60-70 gr/unit, 30-60 gr/unit, 50-70 gr/unit, 40-60 gr/unit. According to a specific embodiment, the fruit diameter is 3-5 cm when fully ripe and prior to natural dehydration. Other exemplary diameters include but are not limited to 3.5-5 cm, 4-5 cm, 3-4 cm and 3.5-4.5 cm. According to a specific embodiment, the processing tomato plant of the present invention is a non-transgenic plant. According to another embodiment the processing tomato plant of the invention is a transgenic plant. The plant may be genetically modified to express genes which are of agronomical value such as increased resistance to insects, nematodes and diseases or increased tolerance to abiotic stress such as low nitrogen, heat, cold, drought etc.

Processing tomatoes are typically grown unstaked in beds, often in double rows. According to a specific embodiment, the processing tomato of the present invention is of the determinate type. This means that the plants complete their effective flowering stage in a set period of plant development, allowing for a concentrated fruit ripening and a single harvest. Determinate plants have a clearly defined growing cycle of less than 150 days from transplanting the small plants until harvest. The harvesting can be done manually or mechanically. Processing tomato plants used for industrial purposes are preferably grown as a bush on the ground (not as a cordon).

As used herein, the phrase “intact skin” refers to an intact tomato fruit which has not undergone any mechanical, physical or chemical treatment which aim to increase cuticular water permeability. Examples of such treatments include, but are not limited to, mechanical treatment such as perforating the skin such as by cutting the skin to increase effectiveness of sun-drying and chemical treatments such as by using agents such as organic solvents that remove the epicuticular wax layer.

The tomatoes may be treated or coated with chemicals such as pesticides, preservants or sulfur, which ensures that the tomatoes retain their natural brilliant red color and a sweet intense flavor; and, also act as a pesticide.

As used herein the term “Brix” which is also referred to as “Degrees Brix” (symbol ° Bx)” is a measure of the percent total soluble solids in a given weight of plant juice, which include the summation of sucrose, fructose, vitamins, amino acids, protein, hormones and other solids. It is often expressed as the percentage of sucrose. It is measured with a saccharimeter that measures specific gravity of a liquid or more easily with a refractometer or a Brix hydrometer.

According to a specific embodiment of the invention the Brix value of the tomatoes is determined using a refractometer. Methods of determining the Brix value of a tomato employing a refractometer are generally known in the art. For comparing compositions of different products having different Brix values, said products may first be brought to the same Brix level by concentration or dilution and subsequently be analysed or, alternatively, said different products may be analyzed at their respective Brix values and the data may subsequently be corrected for the difference in Brix value. Both options are common and well known in the art. In this document, any statements regarding the contents of the tomatoes and tomato pastes will include the respective Brix value, unless it is on a dry solids weight basis.

As used herein the phrase “natural dehydration” refers to the loss of water content of the tomato fruit by evaporation when allowed to dehydrate in-situ (field or green-house) at ambient temperatures such as by sun-drying. Natural dehydration specifically excludes oven drying. The term natural dehydration is thus defined as wrinkling of the intact skin of the tomato fruit post ripening which is generally unaccompanied by bacterial spoilage. Typically, natural dehydration is achieved without man-made cuts through the tomato skin. Natural dehydration involves removal of between 15-60% of the water originally present in the fully ripe tomato, other exemplary ranges include but are not limited to 5-30%, 5-20%, 5-10%, 10-50%, 10-40%, 10-30%, 10-20%, 40-50% or 30-50%. According to a specific embodiment, natural dehydration involves removal of at least 20% of the water originally present in the fully ripe tomato.

The phrase “generally unaccompanied by bacterial spoilage” refers to less than 10³ cfu/gr.

Of note, any reference to a tomato fruit characterized by the present traits, is meant to cover a plurality of plants (or fruits) of which at least 50% of the lot exhibits these traits (e.g., Brix values and optionally lack of bacterial spoilage, as described herein).

Natural dehydration can take place prior to harvest, i.e., by allowing the tomato fruit to remain on the vine post ripening for a time sufficient to reach the desired Brix value. Typical conditions for natural dehydration require sufficiently low humidity (e.g., 10-40%, 15-25% or e.g., 20%) to allow evaporation through the intact skin and ambient temperatures, Typically, natural dehydration is effected by sun-drying. According to a specific embodiment, natural dehydration is effected past the normal harvesting stage when over 90% of the fruit are ripe but not over-ripe. Contemplated exemplary time ranges include but are not limited to 5-21 days, 5-15 days, 5-10 days, 2-18 days, 10-20 days post ripening. The skilled artisan would be able to calculate the dehydration time based on local climate, the tomato cultivar and the end use. According to a specific embodiment, naturally dehydration is effected under low humidity conditions such as below 40% humidity, below 30% humidity and even below 20% humidity.

As used herein the term “ripening” refers to vine ripening (as opposed to ethylene-induced ripening) and is defined as the time when the tomato starts to turn red.

As used herein the phrase “fully ripe” refers to tomatoes having bright red color which is not accompanied by bacterial spoilage dehydration.

To expedite the natural dehydration process, the vine may be cut off the roots so as to prevent water uptake from the soil. According to this embodiment, the fruits remain on the vine, but in the absence of water uptake by the roots, the natural dehydration process is expedited.

Yet alternatively or additionally, natural dehydration can take place following harvest of the tomato fruits (off the vine).

According to a specific embodiment, natural dehydration is effected until a Brix value of up to 30 is reached.

According to a specific embodiment, the Brix value is 12-30.

According to a specific embodiment, the Brix value is 12-15.

According to a specific embodiment, the Brix value is 12-20.

According to a specific embodiment, the Brix value is 15-30.

According to a specific embodiment, the Brix value is 15-25.

According to a specific embodiment, the Brix value is 18-25.

According to a specific embodiment, the Brix value is 17-25.

According to specific embodiments, when the fruit is allowed to remain on the vine, natural dehydration reaches a Brix value above 7.1, which may span the following exemplary ranges of 7.2-20, 7.2-15, 7.2-10, 8-18 or 10-15.

According to a specific embodiment of the invention, the tomato fruit is dehydrated.

According to a specific embodiment, the tomato fruit is naturally dehydrated.

According to a further specific embodiment, the tomato fruit is fully ripe but not subjected to natural dehydration.

According to a further specific embodiment, the tomato fruit is unripe.

According to a specific embodiment, the fruit has a Brix value lower than 7 or lower than 5 (e.g., 5-7, 4.5-7, 4.5-6 or 4.5-5) when fully ripe and prior to being naturally dehydrated.

It is envisaged that the dehydrated tomato fruits of some embodiments of the invention are characterized by good taste and flavor. This is because the tomatoes are naturally dehydrated and not oven-dehydrated, thus avoiding the off-flavors associated with oven drying. The effects of natural dehydration on taste and/or flavor can be evaluated by tasting of the tomatoes and/or paste by one or more test subjects and comparing it with a control. Blind taste testing can be performed to evaluate the taste of the tomatoes or processed products derived therefrom.

Tomato plants of the present invention can be generated by classical breeding methods. A key feature is the selection of plants having fruits exhibiting a Brix value above 7.1 when allowed to naturally dehydrate on the vine, or even above 10 when allowed to naturally dehydrate post ripening and optionally harvesting (exemplary Brix ranges are provided hereinabove).

Thus, processing tomatoes of some embodiments of the invention are generated by crossing a processing tomato plant with a wild Lycopresicon spp. so as to obtain hybrid plants; and, as mentioned, selecting a plant having fruits exhibiting the above indicated Brix values under natural dehydration conditions.

As used herein the term “Lycopersicon spp.” refers to a wild species of the Lycopersicon genus (presently classified as part of the Solanum genus comprising the tomato clade) which normally expresses the CWP polypeptide, as described in Hovav et al. These include the green-fruited species of the genus and an example of such a Lycopersicon spp. is Solanum habrochaites, previously referred to as Lycopersicon hirsutum.

As used herein the term “CWP gene” refers to the nucleic acid sequence encoding the cuticular water permeability gene as described in WO2006/030445 (e.g., GenBank Accession Number DQ840644, SEQ ID NO: 1).

As used herein the term “CWP polypeptide” refers to the expression product of the CWP gene which increases cuticular water permeability (cwp). According to a specific embodiment the CWP polypeptide is that encoding the CWP polypeptide of Lycopersicon hirsutum i.e., GenBank Accession Number: DQ840644.1, SEQ ID NO: 2 or a functional homologue of same as described in WO2006/030445. Such a functional homologue is at least 70%, 80%, 85%, 90%, 95% identical to SEQ ID NO: 2 as determined by global alignment.

In L. hirsutum, the CWP gene is localized to chromosome 4 between the telomeric marker TG464 and the centromeric marker CT173. In the publically available tomato genome sequence this locus is listed as Solyc04g082540 and is located at the chromosomal location SL2.40ch04:63777368 . . . 63779533, as described on Solgene web site.

Exemplary lines of Lycopersicon spp. which can be used according to the present teachings include, but are not limited to LA3935 (line TA517), LA3937 (line TA1473).

The choice of variety of the processing tomato background depends on market demand, regional adaptability, disease resistance and the end use of the product. For example, the Roma type is typically used for drying, while tomato paste processors require a product with high total soluble solids (TSS).

Exemplary cultivars of processing tomatoes include, but are not limited to, SUN 6366, AB 2, Heinz 9780, Heinz 9557, Halley 3155 and Hypeel 303.

Other examples include, the industry tomato (e.g., M82 line) or San Marzano plum.

The development of hybrids in a plant breeding program requires, in general, the development of lines, the crossing of these lines, and the evaluation of the crosses. Most plant breeding programs combine the genetic backgrounds from two or more inbred lines or various other broad-based sources, or mutations into breeding pools from which new inbred lines are developed by selfing and selection of desired phenotypes. Hybrids can also be used as a source of plant breeding material or as source populations from which to develop or derive new plant lines. The expression of a trait in a hybrid may exceed the midpoint of the amount expressed by the two parents, which is known as hybrid vigor or heterosis expression.

Inbred lines may for instance be derived from hybrids by using said methods as pedigree breeding and recurrent selection breeding. Newly developed inbreds are crossed with other inbred lines and the hybrids from these crosses are evaluated to determine which of those have commercial potential.

Pedigree breeding is a system of breeding in which individual plants are selected in the segregating generations from a cross on the basis of their desirability judged individually and on the basis of a pedigree record.

Recurrent selection is a breeding method based upon intercrossing selected individuals followed by continuing cycles of selection and intercrossing to increase the frequency of desired alleles in the population.

Recurrent selection may for instance be performed by backcross breeding, which involves a system of breeding whereby recurrent backcrosses are made to one of the parents of a hybrid, accompanied by selection for a specific character or characters.

A general description of breeding methods commonly used for acquiring different traits in various crops, can be found in reference books such as e.g., Allard, R. W. (1960) Principles of Plant Breeding; Simmonds, N. W. (1979) Principles of Crop Improvement; Mark J. Basset, (1986, editor), Plant Breeding Perspectives; Fehr, (1987) Principles of Cultivar Development Theory and Technique), Curah L (1986) Leek breeding: a review J Hort Sc 61: 407-415.

Thus, the present teachings further comprise selecting for a tomato plant following the step of crossing that is able to naturally dehydrate to the above mentioned Brix values.

According to a specific embodiment of the invention the processing tomato obtained according to the present teachings comprises an introgression which comprises a CWP nucleic acid sequence which is actively transcribed and translated to yield a functional CWP polypeptide or a transgene encoding the CWP polypeptide (typically under transcription regulation of a heterologous promoter).

Thus, according to an embodiment of the invention, the introgression comprises a portion of chromosome 4 of Lycopersicon hirsutum.

According to a specific embodiment, the portion of chromosome 4 of Lycopersicon hirsutum is smaller than a chromosomal fraction extending from telomeric marker TG464 to centromeric marker CT173, Solyc04g082520.

According to a further embodiment of the invention there is provided a processing tomato fruit having a Brix value higher than 7.1 or 10 when subjected to natural dehydration, wherein representative sample of seeds of said processing tomato were deposited at the NCIMB Ltd. (Ferguson Building Craibstone Estate, Bucksburn. Aberdeen Scotland) under the Budapest treaty under XXX (line 1027) or XXX (line 1028).

Seeds of the processing tomato fruit, a sample of such seeds having been deposited under the Budapest treaty, are also contemplated herein.

Plants selected according to the present teachings produce fruits characterized by the aforementioned Brix values under natural dehydration. It will be appreciated by the skilled in the art that Brix values of individual naturally dehydrated fruits may be outside the recited ranges. However, according to a specific embodiment, at least 10%, 25% or more preferably 50% of the dehydrated tomatoes have a Brix value within the specified ranges.

The processing tomatoes of the present invention are typically grown by transplanting but direct seeding is also possible. Transplanting simplifies seedbed preparation and stand establishment, reduces weed competition, provides more options for weed control, and reduced hand-weeding expense. The harvesting is typically effected at once e.g., when at least 50% of the crop exhibits the desired Brix values. The harvesting can be done manually or mechanically, however the latter is more typical to the processing tomatoes industry.

Natural dehydration according to the present teachings is achieved by leaving the fruits on the vine (either harvested or not) prior to or following harvesting or off the vine under ambient temperature conditions e.g., sun-drying.

Fruits are qualified for fruit color, Brix, pH, sugars, organic acids and defect levels (insect damage, mold, green fruit, etc.) at ripening and alternatively or additionally post natural dehydration. The tomatoes are typically transported to a large processing facility, where they are collected and where they may subsequently be washed, typically using chlorinated water and rinsed using tap water and further selected to remove those that present defects (e.g., inadequate ripening, disease damage, molds etc.). Tomatoes may then be processed into a wide variety of products.

For juice or pulp production, the dehydrated tomatoes are comminuted and macerated (disintegrated and broken) to obtain a pumpable mass. As will be clear to the skilled person these operations per se are known and common in the field of tomato processing and any adjustments to the method can be made in this regard without departing from the scope of the invention.

Methods for processing tomatoes and/or producing tomato-based compositions are well known in the art, see generally U.S. Pat. No. 6,924,420. Also reported are specific methods for preparing, for example, paste (U.S. Pat. No. 7,074,451), sterile paste (U.S. Pat. No. 4,206,239), puree (U.S. Pat. No. 4,556,576), sauce (U.S. Pat. No. 7,122,217), solidified sauce (U.S. Pat. No. 4,038,424), barbecue sauce (U.S. Pat. No. 6,869,634), salsa (U.S. Pat. No. 5,914,146), ketchup (U.S. Pat. No. 6,689,279), tomato fiber composition (U.S. Pat. No. 7,166,315) and dehydrated tomato-product (U.S. Pat. No. 5,035,909). Methods of modifying the texture and consistency of tomato paste, pulp, and puree has also been reported, see, for example, U.S. Pat. No. 6,720,019.

Thus, according to an aspect of the invention there is provided a method of producing an edible processed tomato product comprising:

subjecting the processing tomato of the present invention to natural dehydration so as to obtain dehydrated processing tomatoes; and subsequently comminuting or macerating the dehydrated processing tomatoes, thereby producing the edible processed tomato product.

Also provided is an edible processed tomato product comprising the processing tomato of the present invention or an edible portion thereof (e.g., fruit or an edible part thereof).

Also provided is a tomato paste generated according to the present teachings.

Examples of such edible products include, but are not limited to, canned tomatoes (whole), a tomato paste, a ketchup, a tomato sauce a tomato soup, a tomato juice, a tomato powder, a tomato dice, a crushed tomato, a chopped tomato and a tomato concentrate.

It is envisaged that these products comprise the DNA of the processing tomatoes of the present invention.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Production of Industry Tomatoes which Dehydrate on the Vine

Breeding lines of dehydrating tomatoes of indeterminate growth habit (described in U.S. Pat. No. 7,119,261, line 1730 and selfed lines derived from it) were hybridized with the open pollinated determinate industry tomato variety M-82 (LA2838A). From the F2 population derived from this cross, determinate plants homozygous for the cwp allele of the wild species (chosen by molecular marker as described below, as well as by the microfissuring phenotype) were selected. Five of these plants were allowed to self pollinate and F3 plants were grown from each and crossed with 3 elite breeding lines of tomatoes developed for paste production. These breeding lines were similar in type to M-82, were characterized by determinate growth habit and concentrated harvest, characteristic of tomatoes used for paste production. The elite breeding lines differed in fruit shape, from globose to elongated (“Roma” type) and fruit size of from 60 to 90 grams.

Hybrid F1 (BC1F1) plants were grown and selfed seed was collected for the F2 (BC1F2) generation. F1 plants were also backcrossed to the recurrent elite breeding lines for the production of BC2F1 seeds. BC1F2 and BC2F1 plants were grown in screenhouses according to standard cultivation protocols for determinate, concentrated harvest paste tomatoes. Individual BC1F2 and BC2F1 plants, harboring the cwp dehydration allele as determined by molecular marker analysis were selected based on the characteristics of determinate habit, concentrated yield (at least 20 ripe fruit per plant) and dehydration trait indicated by microfissures and wrinkling of the ripe fruit.

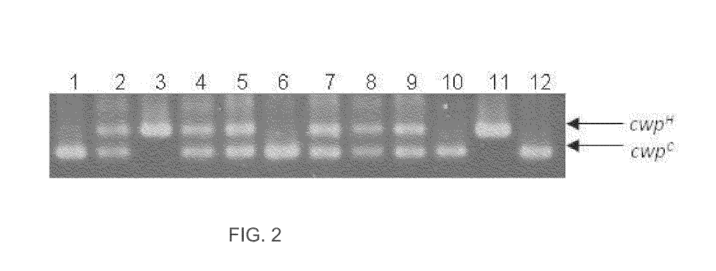

The cwp marker analysis was performed by detecting the deletion located in the third intron of the CWP gene in lycopersicum. Primer cwp forward CGTACTCAAACGATGATAAAGGT (SEQ ID NO: 3), Primer cwp Reverse TTATTGCATTTGGAGTTTTTCAATCCG (SEQ ID NO: 4). PCR conditions consisted of Tm 56 degrees, elongation time about 15 s, 35 cycles. Results of such a molecular analysis are shown in FIG. 2.

At least 20 ripe fruit of individual selected plants were harvested, immediately weighed and Brix values of the pulp extracted from 5 fruit at harvest were measured, using an Atago A-1 digital refractometer. The remaining fruit were allowed to remain in a non-heated room (11° C. min and 24° C. max in Bet Dagan, Israel). After 7 days additional 5 fruits were removed for fresh weight and Brix measurements. At 14 days the remaining fruit (at least 10) were weighed and pulp was extracted using a blender and passed through a 2 mm-mesh sieve to remove seeds and skin. The extracted pulp was measured for Brix as above. Samples of the pulp were weighed and dried in an oven to determine water and dry matter contents of the paste. Samples of the pulp were tasted and found to be very tasty. Additional samples were weighed and frozen and used for chemical analyses.

Results

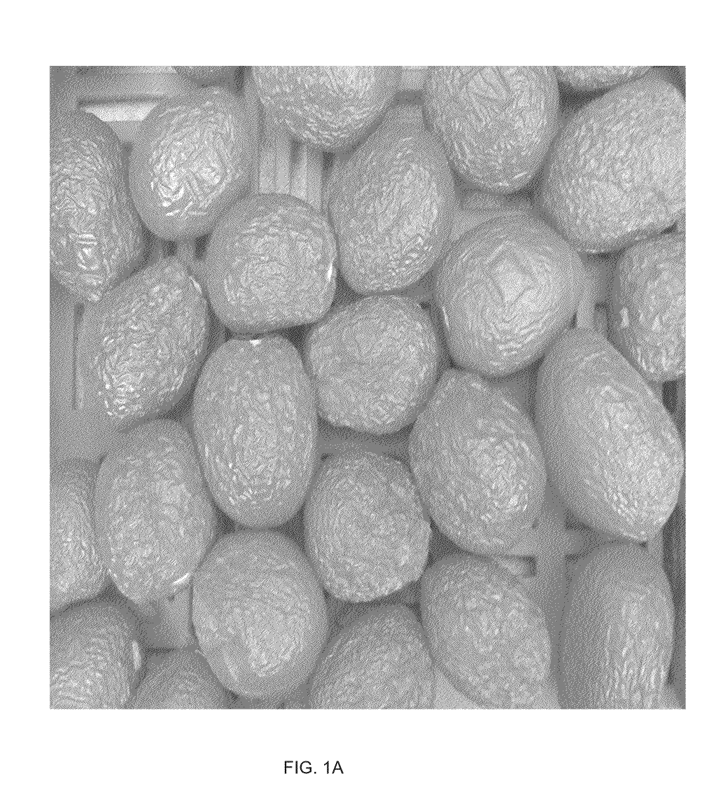

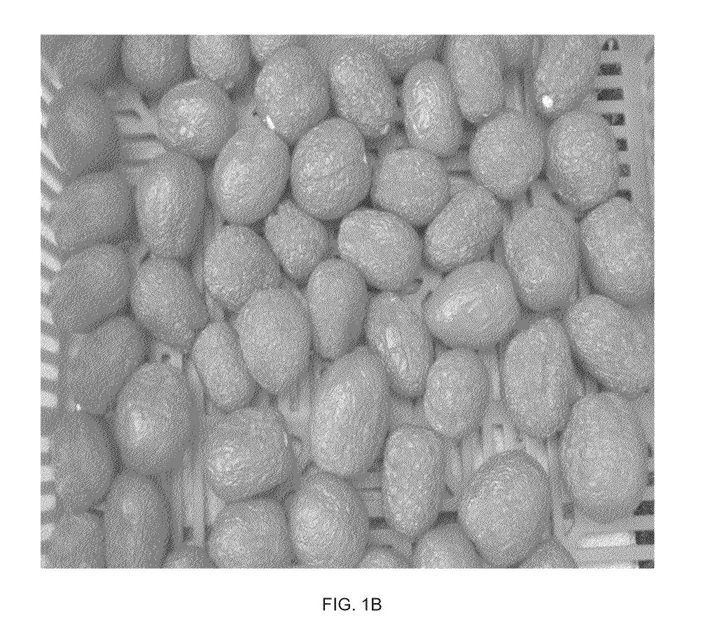

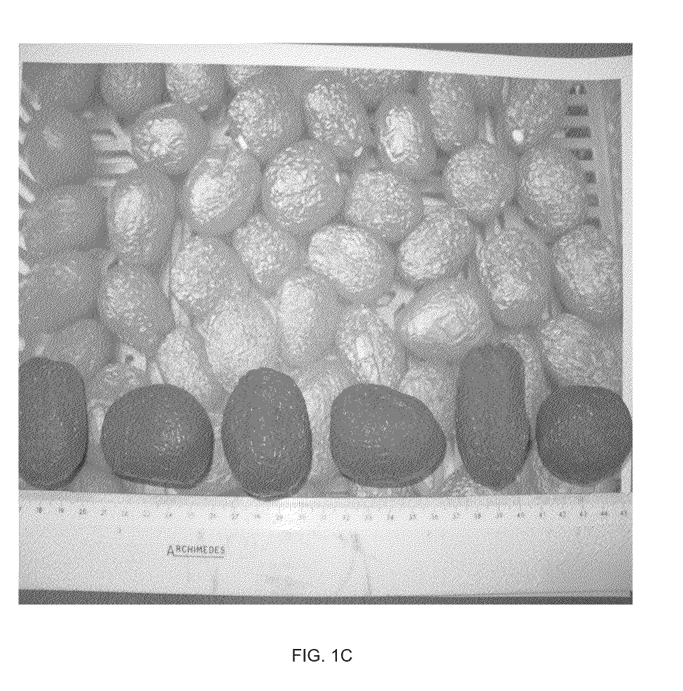

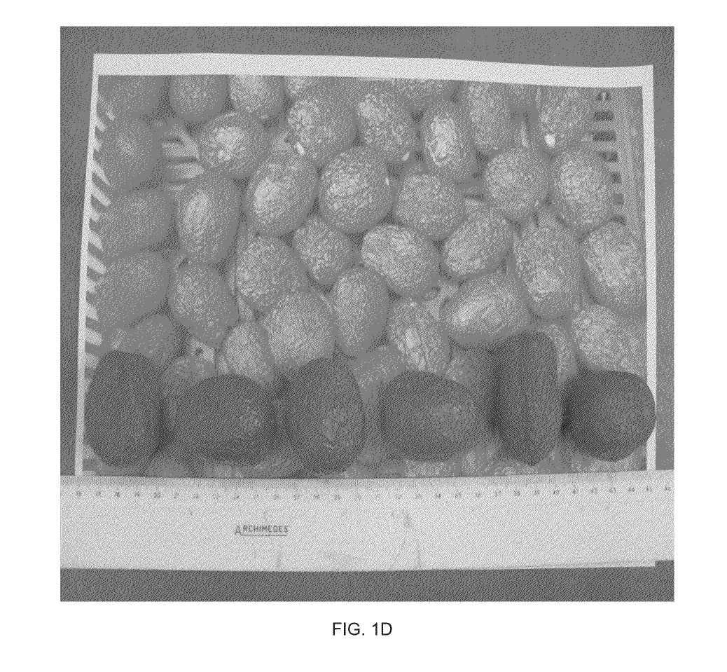

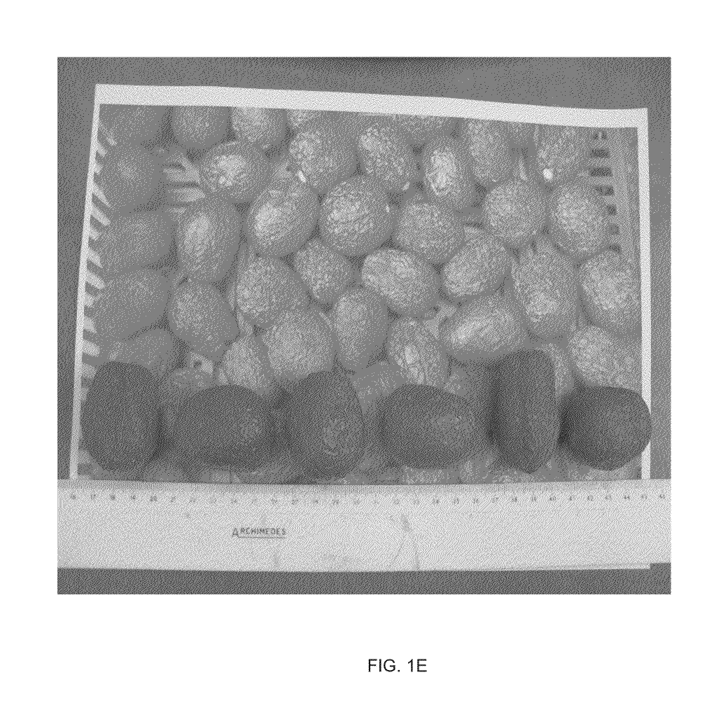

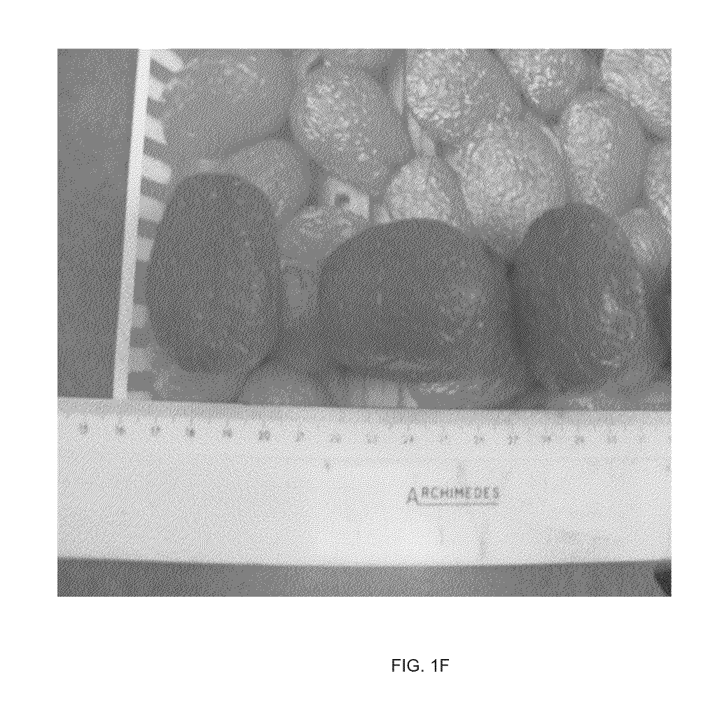

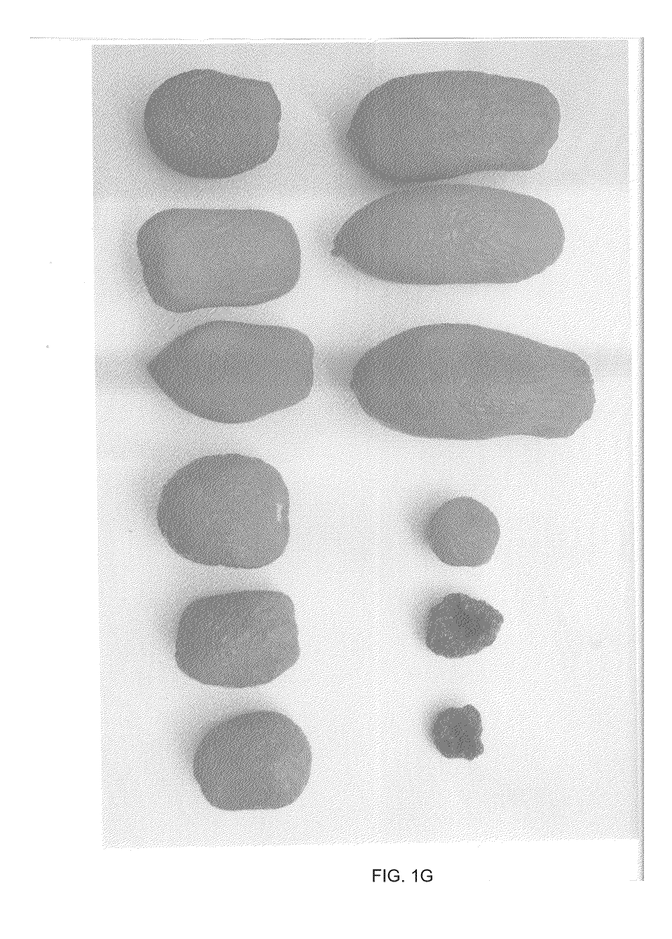

Table 1 below shows the Brix and pH values of the pulp produced naturally dehydrated tomato fruit from selected F2 and BC1 plants. The table shows that, for example, fruit of over 60 gram reached a Brix of over 10 (14-145), of over 50 gram reached Brix of over 12 (15-101), and of over 40 gram reached brix of over 15 (15-159). Pulp of these plants had % dry weights of 11.4, 14.4, and 16. In addition pH values were generally above 4.6, significantly less acidic than standard tomato pulp, as shown for the control variety (pH 4.4, Brix 4.9). FIGS. 1A-G describe of BC2F1 plants true hybrids as they are ˜75% the industry type recurrent parent.

TABLE 1

| at | | | | | |
|---|---|---|---|---|---|
| harvest | fruit fw (gm) | Brix | pH | % dry weight | pulp water content |
| control | 65 | 4.9 | 4.4 | 5.7 | 94.3 |

| | Pulp at | | Pulp after | | | | | |
|---|---|---|---|---|---|---|---|---|
| | harvest* | | 7 days** | | Pulp after 14 days*** | | | |
| | fruit fw | | fruit fw | | | | % dry | water |
| plant | (gm) | Brix | (gm) | Brix | Brix | pH | weight | content |
| 14-58 | 63.2 | 5.7 | 39 | 5.7 | 7.8 | 4.9 | 8.7 | 91.3 |
| 14-167 | 57.0 | 4.7 | 38 | 4.3 | 6.8 | 4.8 | 7.8 | 92.2 |
| 14-145 | 64.0 | 6.6 | 51 | 6.8 | 10.5 | 4.7 | 11.4 | 88.6 |
| 14-166 | 65.4 | 4.9 | 46 | 5.6 | 9.1 | 4.7 | 10.7 | 89.3 |
| 15-101 | 53.4 | 7 | 36 | 9.4 | 12.6 | 4.8 | 14.4 | 85.6 |
| 18-88 | 47.6 | 7.5 | 36 | 7.8 | 7.2 | 4.6 | 7.9 | 92.1 |
| 14-18 | 54.2 | 5.1 | 40 | 5.9 | 8.0 | 4.6 | 9.8 | 90.2 |
| 15-159 | 41.8 | 6.6 | 29 | 9.2 | 15.7 | 4.8 | 16.0 | 84.0 |
| 21-13 | 41.4 | 6.4 | 28 | 6.2 | 9.1 | 4.8 | 10.3 | 89.7 |
| 17-125 | 40.8 | 6.9 | 29 | 8.7 | 11.7 | 4.9 | 12.9 | 87.1 |
| 16-132 | 34.8 | 7.2 | 23 | 8.7 | 15.4 | 4.8 | 18.0 | 82.0 |
| 14-131 | 35.8 | 5.5 | 25 | 5.8 | 8.4 | 4.7 | 9.6 | 90.4 |
| 15-134 | 40.8 | 8 | 29 | 8.7 | 14.8 | 4.9 | 16.2 | 83.8 |
| 17-1 | 34.0 | 8.2 | 22 | 11.2 | 17.0 | 4.8 | 17.9 | 82.1 |

*At harvest-on the vine at full ripe
*,**pulp after 7 days or 14 days following harvest under natural dehydration of the tomato having an intact skin

Analysis on further generations of breeding was done on tomato fruit grown in the Arabah, Israel during the cold season.

Lines of different generations of breeding included BC1F3, BC3F2 and BC2F3 lines. Of note, generally, the number of BC indicates the number of times the material was crossed with the industry type recurrent parent. The BC1F3 is lines that were selfed 3 times after an initial cross of our parental donor lines with the industry line; BC3F2 is lines that were selfed twice after 3 crosses with the industry line; BC2F3 is lines that were selfed three times after 2 crosses with the industry line.

All of the lines described have fruit size of at least 30 grams fresh weight at harvest and a Brix of at least 7. They are all determinate and suitable for once-over mechanical harvest. As shown in Table 2 below, the weight decreases and the brix increases concomitantly following a week at room temperature (25° C.).

TABLE 2

| | time 0 | | | | | |
|---|---|---|---|---|---|---|
| | average FrW | time 0 | 7 days ambient | 7 days ambient | % change fw | % change TSS |
| line | gr | TSS (brix) | average FrW gr | TSS (brix) | 7 days | 7 days |
| AB-2: | 102 | 4.2 | 88 | 4.0 | −14 | −5 |
| control | | | | | | |
| 24-3 | 40 | 10.9 | 26 | 12.4 | −34 | 14 |
| 5-159 | 37 | 9.8 | 19 | 15.6 | −49 | 59 |
| 8-2 | 34 | 9.6 | 24 | 11.4 | −30 | 19 |
| 5-151 | 31 | 9.3 | 22 | 12.2 | −28 | 31 |
| 24-1 | 54 | 8.4 | 36 | 11.2 | −33 | 33 |
| 16-2 | 38 | 8.1 | 23 | 10.0 | −39 | 23 |
| 4-115 | 43 | 7.5 | 32 | 10.3 | −26 | 37 |
| 5-141 | 39 | 7.5 | 19 | 15.6 | −52 | 108 |
| 10-256 | 53 | 7.4 | 38 | 8.5 | −28 | 15 |

Clearly, harvested fruits of all the lines tested exhibited a Brix value higher than 8 at room temperature when left to dehydrate for 7 days untreated.

Hybrid lines show TSS above 7 upon ripening and size of 30 g at harvest, results not shown.

### Example 2

### Pedigree of Deposited Lines

Line 1047:

Dehydrating parental line 5607 was crossed with a typical industry tomato breeding line M120G. Dehydrating parental line 5607 is a BC2F3 line derived from the cross of a parental line homozygous for the cwp dehydration allele, backcrossed for two consecutive crosses to the San Marzano tomato variety for the purpose of introducing the elongated plum fruit shape to the line. The industry tomato breeding line M120G is characterized by the following traits: determinate growth habit, uniform shoulders, jointless, red fruit, oblong shape, Brix of 4.5-5.0, resistant to I2. F1 plants were backcrossed to the recurrent breeding line M120G and F3 plants from this cross were genotyped for the cwp gene, resulting in line 1047.

Fruits were harvested from a plot of 24 plants of line 1047 and brought to the laboratory where a sample of the fruit was weighed and Brix measured by refractometer. At harvest fruit showed signs of dehydration as evidenced by wrinkling of the skin. Fruit of this line averaged 38 grams at harvest with a Brix of 8.1. The remaining fruit was allowed to remain in the laboratory at a temperature of 25° C. for an additional 7 days after which weight and Brix were measured. Weight was an average of 23 grams and a Brix of 10.0, for a loss of 39% fresh weight and an increase of 23% Brix.

Line 1048

Dehydrating parental line 5600 was crossed with a typical industry tomato breeding line 7367. Dehydrating parental line 5600 is a BC2F4 line derived from the cross of a parental line homozygous for the cwp dehydration allele, backcrossed for two consecutive crosses to the M-82 industry tomato variety. The industry tomato breeding line 7367 is characterized by determinate growth habit, uniform shoulders, jointless, dark red fruit, elongated shape, resistant to Ve, I2, N. F1 plants were backcrossed to the recurrent breeding line 7367 twice in two consecutive seasons and F2 plants from this last cross were genotyped for the cwp gene, resulting in line 1048.

Fruit were harvested from a plot of 24 plants of line 1048 and brought to the laboratory where a sample of the fruit was weighed and Brix measured by refractometer. At harvest fruit showed signs of dehydration as evidenced by wrinkling of the skin. Fruit of this line averaged 54 grams at harvest with a Brix of 8.4. The remaining fruit was allowed to remain in the laboratory at a temperature of 25 C for an additional 7 days after which weight and Brix were measured. Weight was an average of 36 grams and a Brix of 11.2, for a loss of 33% fresh weight and an increase of 33% Brix.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A processing tomato fruit having an intact skin and a Brix value higher than 10 under conditions for natural dehydration, which do not include oven dehydration, said natural dehydration being generally unaccompanied by microbial spoilage, said processing tomato fruit weighing 30-80 gr when fully ripe and prior to said natural dehydration, wherein the processing tomato fruit comprises a wild species allele of cwp allowing said natural dehydration and further wherein said natural dehydration is in an open field.

2. A processing tomato fruit having an intact skin and a Brix value higher than 7.1 under conditions for natural dehydration, which do not include oven dehydration, said natural dehydration being generally unaccompanied by microbial spoilage, wherein said conditions for natural dehydration comprise allowing the processing tomato fruit to remain on the vine post ripening for a time sufficient to reach said Brix value, said processing tomato fruit weighing 30-80 gr when fully ripe and prior to said natural dehydration, wherein the processing tomato fruit comprises a wild species allele of cwp allowing said natural dehydration and further wherein said natural dehydration is in an open field.

3. The processing tomato fruit of claim 1, wherein said conditions for natural dehydration comprise allowing the processing tomato fruit to remain on the vine post ripening for a time sufficient to reach said Brix value.

4. The processing tomato fruit of claim 1, wherein said conditions for natural dehydration comprise sun-drying prior to or following harvest of the processing tomato fruit.

5. The processing tomato fruit of claim 1 being dehydrated.

6. The processing tomato fruit of claim 1 being naturally dehydrated.

7. The processing tomato fruit of claim 1 being fully ripe and not subjected to dehydration.

8. The processing tomato fruit of claim 1 being unripe.

9. The processing tomato fruit of claim 1, wherein said natural dehydration is defined as wrinkling of said intact skin of the processing tomato fruit post ripening following harvesting.

10. The processing tomato fruit of claim 1, characterized by an intact skin which permits dehydration of the fruit so as to obtain wrinkling of the skin.

11. The processing tomato fruit of claim 1, wherein the genome of said processing tomato fruit comprises a nucleic acid sequence encoding the cwp polypeptide, wherein said cwp polypeptide causes increased water permeability.

12. The processing tomato of claim 11, wherein said nucleic acid sequence is under a transcriptional control of a heterologous promoter.

13. The processing tomato of claim 11, wherein said nucleic acid sequence forms a part of an introgression derived from a wild Lycopersicon spp. said introgression comprising a portion of chromosome 4 of said Lycopersicon spp.

14. (canceled)

15. The processing tomato of claim 13, wherein said Lycopersicon spp. is Lycopersicon hirsutum.

16. The processing tomato of claim 1, weighing 40-80 gr when fully ripe and prior to said natural dehydration.

17. (canceled)

18. The processing tomato of claim 1, wherein said Brix value is 12-30.

19. The processing tomato of claim 1, wherein said Brix value is 12-25.

20. The processing tomato of claim 1, wherein said Brix value is 12-20.

21. The processing tomato of claim 1, having a Brix value lower than 5 when fully ripe and prior to said natural dehydration.

22. A plant comprising the fruit of claim 1.

23. A seed of the tomato fruit of claim 1.

24-25. (canceled)

26. A method of producing a tomato paste comprising:
subjecting the processing tomato of claim 1 to natural dehydration so as to obtain dehydrated processing tomatoes; and subsequently comminuting or macerating said dehydrated processing tomatoes, thereby producing a tomato paste.

27. A tomato paste generated according to the method of claim 26.

28. An edible processed tomato product comprising the processing tomato of claim 1 or an edible portion thereof.

29. (canceled)

30. A method of producing the tomato of claim 1, the method comprising:
crossing a processing tomato plant with a wild Lycopersicon spp. so as to obtain hybrid plants; and
selecting plants of said hybrid plants having fruits exhibiting a Brix value above 7.1 when subjected to natural dehydration without oven dehydration, thereby producing the tomato of claim 1.

31-33. (canceled)

34. The plant of claim 22 being an inbred.

35. The plant of claim 22, being a hybrid plant.


SEQUENCE LISTING (4 sequences)

SEQ ID NO: 1 - DNA, 3153 bp, Solanum habrochaites
  misc_feature (1500)..(1500): n is a, c, g, or t
tgccgtcctattcttagaatactcaagtaatttaacgtagtggtgaaaatttgataaattaattatatactaatttttcagtcttattttatgtggtatatttaattggatatgtagtttaagaaataataaaaactttaaaatatttataaatttacttttctaaaaaagtgaattcaattttttctctcctcataaatgtattagagtattatcattaaaattaagtgggactaataaaggtaaaaaataaattattcctttaaattatttaaccatataagaaaatgtgacattcttttttagacttgactaaaatagaaaataatgtcatatatataaaatgagacgaaaaaagtaaatattaatttaaaatttaaaactttagggtaatagctactttgaattacctagatttcaataaaattcaacatataataaaacatactaatttacaatttttaaaataatatgactaaaagtcatattattcaaaaaacaatctataccgccgtcacctagttactttaatttgtgtagcttctagtacatacatttttaaactttatctgaatttaatattttaattatattaaacatttattaaaatttataaaatttaaattgacgtaatataatgaagagagtagtactatataaaccatgtgagtactaacatgatcttcatcttattcttgtttttatttatagaaacaataaaatagttataaaattaatcaatcatgtgtatagtagtgtttatttgggaagcagatagtagatattcattagtgttattattgaatagagatgaatatcataataggccaacaaaggaagttcattggtgggaagatggagaaattgttggtggcaaagatgaagttggtggtggcacttggttggcttcttcaactaatggtaaatggtttcttactaatgttttggaaattcatacacttcctcatgccaaaactagaggtgatctacccgttcgatttttacaggtacgattaaattctttatatattatacgttaatatgtttgatctttcattttggttttgttatacgaaggacgagacctagaggtctttaagacaaaacataaatatgcatcatagtcataaactttcaataaatattcaattttgaatatgcgctttcaaaggtattacaagttgagtactaaaggaattgagtttatcaagattaaattttgaatttgattcttttgatcatgattaatagtaatgttaaatcttgtccttattggagtatatatatgatcaataaatcaagattttaaattgtagtataatcttaattttaaagaatattaatgttgtaaaattttagatttaacaaacacaaaaatcatatttgtatgttataactatagtttgtatagttgcgctcaatatgtttgttcgcgagctgttaatatgtcactatttcggtttacatatacaaaagagatcaattgcataattttgtttngcatatacntnttaaacatgatacataatagaaatttcattnattgtgtaatatatctttgtataaagcaagaaagagcgaaacacaacagaaaactggatagggaaatatttatattttgtatagttataagtgtatatgacggaaatatacgtaattattttttatacatgattttctctcgcttttatgcaaacacaaacacaatttatacatttgtttttgtgtaaagtgagagtggcgagcgagattctatagagagagaaccaaatgaaaatatatgtattatatgcagttttctgtagttttatacaaatacaaacacaatttatacatttatttttgtgtatgagagaggcgagtgagattctcnggggaggaaaatatatgtatatatacagttttgtttcgctataaacaaacagaacacattttatacatttgtatttgtataaaacaagagagacgagggagaaactgctcaacgagaaattcaggaagagaggtgaatgacaactatttgttacgagttgcaagtaaatcaaactgcgactataacatttagtttgaattaataatttgttattttaaacgattttccgtaaaatttaattgttaattgcagagcaacaaaagcccaatggagtttgcaaaagagttggtgaatgaagggaatgaatacaatgggtttaatttaattttggcagatattgaaactaaaaaaatggtatatgtaacaaataggcccaaaggagagcccataacaatacaagaagtccaaccaggtattcatgtgctgtccaatgcaaaactggactctccttggcccaaggtaagaattctaatgggcttttttcgatcgatatacataaattatacaaatgatatgcttttggttgttcatttcaggctcaaagactgaagttaaattttaagaaaatgatggatgtttacgaagtgaatgacgagaaaatctgcgtcaaagatatgatagaaaaattgatgagagataccactaaagctgataaaagtaaattgccttgtatttgttctacagactgggagttggaacttagctctattttcgtggaagttgacactcactgggtaattcataccgcgttataactaatatgtttgtttgattttaacgtactcaaacgatgataaaggttaaagtagatatacaaacattttaaaaataattgaaatagttcaataatagaagtgtacatatcattaacatagtttgatgggtttttttggtggtgtgaatatgtaggggtgttatggtactagaagtacaacagcattgacgattgaagtgggaggagaagtaagcttttatgagttgtaccttgagaacaacatgtggaaagagcaaattgtcaactatcggattgaaaaactccaaatgcaataaatgtttttcatatgttgacatatctaatgtaaatgtttttcatatgttgacatatctaatgttttcattttttttttttaattcaaataagattttttcttcaaaaaattaaactttttgtctttgaatggaaattgttattcattgtatttgtaaaatgtactacactacttggaagacataatgtatgtttcgggctcctttgttttagcaacaattttagactttca

SEQ ID NO: 2 - protein, 269 aa, Solanum habrochaites
MCIVVFIWEADSRYSLVLLLNRDEYHNRPTKEVHWWEDGEIVGGKDEVGGGTWLASSTNGKWFLTNVLEIHTLPHAKTRGDLPVRFLQSNKSPMEFAKELVNEGNEYNGFNLILADIETKKMVYVTNRPKGEPITIQEVQPGIHVLSNAKLDSPWPKAQRLKLNFKKMMDVYEVNDEKICVKDMIEKLMRDTTKADKSKLPCICSTDWELELSSIFVEVDTHWGCYGTRSTTALTIEVGGEVSFYELYLENNMWKEQIVNYRIEKLQMQ

SEQ ID NO: 3 - DNA, 23 bp, Artificial sequence
  Single strand DNA oligonucleotide
cgtactcaaacgatgataaaggt

SEQ ID NO: 4 - DNA, 27 bp, Artificial sequence
  Single strand DNA oligonucleotide
ttattgcatttggagtttttcaatccg